(19) United States
(12) Patent Application Publication
(10) Pub. No.: US 2004/0220281 A1
(43) Pub. Date: Nov. 4, 2004

(54) Treatment of hormone-refractory prostate cancer

(76) Inventor: Lorne J. Brandes, Winnipeg (CA)

Correspondence Address: SIM & MCBURNEY, 330 UNIVERSITY AVENUE, 6TH FLOOR, TORONTO, ON, M5G 1R7, CA

(21) Appl. No.: 10/439,302

(22) Filed: May 16, 2003

Related U.S. Application Data
(60) Provisional application No. 60/466,472, filed on Apr. 30, 2003.

(51) Int. Cl. A61K 31/137

(52) U.S. Cl. 514/651

(57) ABSTRACT

A proportion of patients with hormone-refractory prostate cancer have greater survival times by treatment with DPPE, mitoxantrone and prednisone than patients receiving mitoxantrone and prednisone alone.

### REFERENCE TO RELATED APPLICATIONS

This application claims priority under 35 USC 119(e) from pending U.S. Provisional applications No. 60/466,472 filed Apr. 30, 2003 and ______ filed ______.

### FIELD OF THE INVENTION

This invention relates to treatment of prostate cancer.

### BACKGROUND TO THE INVENTION

One of the major chemotherapeutic treatments is that of malignant growth (cancer) in humans. The objective of chemotherapy is the total extermination of clonogenic tumor or malignant cells, with minimal damage to the patient However, one of the major limitations of the chemotherapeutic approach for managing human cancer is the general inability of anticancer drugs to discriminate between normal and tumorous cells Anti-neoplastic agents have the lowest therapeutic indicies of any class of drugs used in humans and hence produce significant and potentially life-threatening toxicities. Certain commonly-used anti-neoplastic agents have unique and acute toxicities for specific tissues. For example, the vinca alkaloids possess significant toxicity for nervous tissues, while adriamycin has specific toxicity for heart tissue and bleomycin has for lung tissue. In general, almost all members of the major categories of anti-neoplastic agents have considerable toxicities for normal cells of gastrointestinal, epidermal and myelopoietic tissues.

Generally, the dose-limiting consideration for chemical management of cancer in humans is the toxicity that anti-neoplastic agents have for the pluripotent stern cells of myelopoietic tissue. This toxicity arises from the fact that most anticancer drugs function preferentially against proliferating cells but with no significant capacity to discriminate between cycling normal and cycling tumor tissues.

In U.S. Pat. Nos. 6,288,799, 5,859,065, 5,708,329, 5,747,543 and 5,618,846, all assigned to University of Manitoba and the disclosures of which are incorporated herein by reference, there is described an improved method for the in vivo chemotherapeutic treatment of cancer in which there is first administered a compound which inhibits normal cell proliferation while promoting malignant cell proliferation, specifically a potent antagonist selective for intracellular histamine receptors, in an amount sufficient to inhibit the binding of intracellular histamine to the receptors in normal and malignant cells. Following sufficient time to permit the inhibition of binding of intracellular histamine, a chemotherapeutic agent is administered. An enhanced toxic effect on the cancer cells from the chemotherapeutic agent is obtained while any adverse effect of the chemotherapeutic agent on normal cells, particularly bone marrow and gastro-intestinal cells, is significantly ameliorated One useful compound which inhibits normal cell proliferation while promoting malignant cell proliferation is N,N-diethyl-2-[4-(phenylmethyl)-phenoxy]ethanamine, abbreviated herein as DPPE.

Brandes et at (1994, 1995) have shown that N,N-diethyl-2-[4-(phenylmethyl)phenoxy]-ethanamine (DPPE, tesmilifene), a tamoxifen analogue and intracellular antagonist of histamine binding to P450, potentiates the cytotoxicity of a variety of antineoplastic drugs in vitro (Brandes et al, 1987) and in vivo (Brandes et at, 1991). DPPE is a potent substrate for CYP 3A4 (Brandes et al., 2000), an isozyme that metabolizes arachidonic acid, hormones and drugs, including many antineoplastic agents Like other substrates for 3A4, DPPE also inhibits the p-glycoprotein (P-gp) pump (Menendez et al. 1998), whose overexpression is implicated in drug resistance

Hormone-refractory prostate cancer is defined by progressive malignancy despite adequate hormonal blockade, confirmed by demonstrably suppressed serum testosterone levels less than 30 ng/ml. Traditionally, this clinical entity has been characterized by symptoms of tumor progression (most often bone pain)

In addition to radiotherapy, cytotoxic chemotherapy has been shown to have anti-cancer efficacy against advanced prostate cancer after failure of hormonal therapy, with agents such as cyclophosphamide, doxorubicin and the taxanes, causing reduction of symptoms in up to 50% of treated cases and objective tumor reduction in 10-20% of cases (Beer and Raghavan, 2002) Mitoxantrone, a well characterized anthracenedione, has been shown to have significant anticancer efficacy against prostate cancer, both in phase II (Osborne et al, 1983; Raghavan et al, 1989) and phase III clinical trials (Tannock et al, 1996; Kantoff et al, 1999). In patients with symptomatic bone metastases, mitoxantrone in combination with prednisone causes a reduction of symptoms in up to 40% of cases (Tannock et al, 1996; Raghavan et al, 1989), PSA response in 30 to 40% and median survival of around 10 months. In series that include asymptomatic patients with bone metastases, the median survival may be as long as 16 to 18 months, depending on the case mix (Kantoff et al, 1999), although stage migration could account for the apparent improvement in survival after chemotherapy in asymptomatic patients. Based on these findings, this agent was approved by the Food and Drug Administration for the management of hormone-refractory prostate cancer.

In a phase II study of DPPE and cyclophosphamide in 20 patients with hormone-refractory prostate cancer, a PSA response rate of 50% and pain response rate of 85% were observed (Brandes et al., 1995). In a phase III prospective randomized trial conducted by the National Cancer Institute of Canada, comparing DPPE/doxorubicin (N=151) to doxorubicin (N=152) in breast cancer patients, DPPE/doxorubicin was shown to have the unusual effect of significantly increasing median survival (23.6 vs. 15.6 months, P<0.03) without a major impact on the early endpoints of response rate and progression-free survival (PCT/CA02/01725 filed Nov. 6, 2002).

### SUMMARY OF THE INVENTION

It has now been surprisingly found, in a Phase II, non-randomized clinical trial of DPPE combined with the standard regimen of mitoxantrone and prednisone in the treatment of hormone-refractive prostate cancer that overall survival in a significant number of patients was increased as compared to the work reported earlier without DPPE pretreatment. The patients exhibiting longevity all had significant pain symptoms and tended to have high PSA and alkaline phosphates levels, indicative of a high disease burden and poor prognosis for survival beyond one year.

Accordingly, in one aspect, the present invention provides a method of achieving enhanced survival in a proportion of human patients with hormone-refractory prostate cancer, which comprises:

(a) first administering to said patients at least one diphenyl compound of the formula:

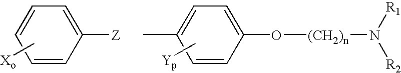

wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each an alkyl group containing 1 to 3 carbon atoms or are joined together to form a heterocyclic ring with the nitrogen atom and n is 1, 2 or 3, or pharmaceutically-acceptable salts thereof, and

(b) following sufficient time to permit inhibition of binding of intracellular histamine, subsequently administering to the patient a chemotherapeutic agent active in hormone-refractory prostate cancer.

In the application of the present invention, the diphenyl compound and the chemotherapeutic agent are generally administered by intravenous infusion. In one preferred procedure, a solution of the diphenyl compound is administered to the patient over a desired period of time prior to administration of the chemotherapeutic agent and a solution of the chemotherapeutic agent in combination with the diphenyl compound then is administered for the period of administration of the chemotherapeutic agent. If desired, a solution of the diphenyl compound is administered after completion of the administration of the chemotherapeutic agent for a desired period of time to ameliorate side effects from the chemotherapeutic agent administration.

### BRIEF DESCRIPTION OF DRAWINGS

FIG. 1 is a graphical representation of the results of the Phase II trial referred to herein, illustrating the estimated probability of survival as a function of months from start of treatment.

### GENERAL DESCRIPTION OF INVENTION

In the present invention, a diphenyl compound is used which is a potent antagonist of histamine binding at the intracellular histamine receptor and is administered in an amount sufficient to inhibit the binding of intracellular histamine at the intracellular binding rate (H_{IC}) in normal cells. Such compounds exhibit a pK₁ of al least about 5, preferably at least about 5.5.

Specific potent compounds which are useful in the present invention are diphenyl compounds of the formula:

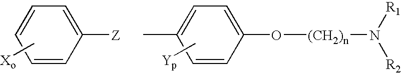

wherein X and Y are each fluorine, chlorine or bromine, Z is an alkylene group of 1 to 3 carbon atoms or ═C═O, o and p are 0 or 1, R₁ and R₂ are each alkyl group containing 1 to 3 carbon atoms or are joined together to form a hetero-ring with the nitrogen atom and n is 1, 2 or 3. Pharmaceutically-acceptable salts of the diphenyl compounds may be employed.

Alternatively, the benzene rings may be joined to form a tricyclic ring in accordance with the structure:

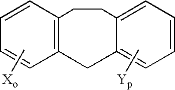

In one preferred embodiment, the group

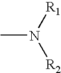

is a diethylamino group, although other alkylamino groups may be employed, such as dimethylamino, and, in another preferred embodiment, a morpholino group, although other heterocyclic ring groups may be employed, such as piperazino. o and p are usually 0 when Z is an alkylene group and o may be 2. In one particularly preferred embodiment, Z is —CH₂—, n is 2, o and p are each 0 and

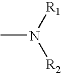

is a diethylamino group. This compound, namely N,N-diethyl-2-[4-(phenylmethyl)-phenoxy]ethanamine, which may be in the form of the free base or in the form of its hydrochloride salt, is abbreviated herein as DPPE. In addition to a methyl group linking the benzene rings, other linking groups may be employed, such as ═C═O. Other substitutents may be provided on the benzene rings in addition to the halogen atoms, for example, an imidazole group.

The diphenyl compound employed in the present invention is administered to the patient in any convenient manner, such as by intravenous injection of a solution thereof in an aqueous pharmaceutically-acceptable vehicle. The diphenyl compound is administered to the patient over a period of time before administration of the chemotherapeutic agent

The chemotherapeutic agent employed herein is one which is active in hormone-refractory prostate cancer. Such chemotherapeutic agents active in cancer include anthancene diones, such as mitoxantrone The chemotherapeutic agent, or a mixture of such agents, is administered in any manner consistent with its normal manner of administration in conventional prostate cancer therapy, often by intravenous infusion of a solution thereof. One specific chemotherapeutic agent which may be used is mitoxantrone, which may be used in combination with prednisone.

The administration of the diphenyl compound to the patient prior to administration of the chemotherapeutic agent is necessary in order to permit the diphenyl compound to inhibit the binding of intracellular histamine in normal and malignant cells and thereby, in effect, shut down the proliferation of the normal cells, but increase proliferation of malignant cells.

The length of time prior to administration of the chemotherapeutic agent(s) that the diphenyl compound is administered depends on the diphenyl compound, its mode of administration and the size of the patient. Generally, the diphenyl compound is administered to the patient for about 30 to about 90 minutes, preferably about 60 minutes, prior to administration of the chemotherapeutic agent(s).

The quantity of diphenyl compound administered to the patient depends on the side effects to be ameliorated, but should be at least sufficient to inhibit binding of intracellular histamine in normal cells. The quantity required to achieve the beneficial effects of the present invention depends upon the diphenyl compound employed, the chemotherapeutic agent(s) employed and the quantity of such agent(s) employed.

In general, the quantity of diphenyl compound employed in humans is from about 8 to about 320 mg/M² of human to which the diphenyl compound is administered, with about 8 and 240 mg/M² being the optimal dose for gastro-intestinal and bone marrow protection, respectively. Over this dose range, the present invention is able to achieve an enhanced chemotherapeutic effect on hormone-refractory prostate cancer cells while, at the same time, also protecting normal cells from damage by the chemotherapeutic agent(s) in a wide variety of circumstances where traditional chemotherapy leads to damage of normal cells or tissues not involved in the disease process.

In the treatment of hormone-refractory prostate cancer, the diphenyl compound preferably is used in an amount of about 3 to about 10 mg/kg of patient administered intravenously over a period of about 30 to about 90 minutes prior to administration of the chemotherapeutic agent(s) and continuing for the period of administration of The chemotherapy agent(s). In the specific Phase II clinical trial described herein, there was employed 5.3 mg/kg of DPPE in the form of the base (equivalent to 6 mg/kg of DPPE in the form of its hydrochloride), administered intravenously as an aqueous solution thereof over 80 minutes, with the last twenty minutes being accompanied by infusion of mitoxantrone.

The treatment of the patients with DPPE and mitoxantrone may include standard therapy with prednisone or the glucocorticoid, such as prednisolone or related cortisone.

### EXAMPLES

### Example I

This Example describes a Phase II clinical trial of patients with hormone-refractory prostate cancer.

A Phase II trial was conducted as a non-randomized, single-arm clinical trial, according to a protocol that was approved by the Clinical Investigations Committees and the Institutional Ethical Review Boards of the University of Southern California and the University of Manitoba. Patients underwent history and physical examination, detailed assessment of hematological and biochemical parameters, bone scans, and CT scans of abdomen and pelvis. Based on prior preclinical and clinical modeling experience (Brandes et al, 1994, 1995, 1996), patients received a 3-weekly regimen (maximum number of cycles, 12) consisting of DPPE (5.3 mg/kg infused intravenously over 80 minutes) and mitoxantrone (12 mg/m² i.v. during the last 20 minutes of DPPE infusion), as well as daily oral prednisone (5 mg bid). To prevent or ameliorate motion sickness associated with the DPPE infusion, patients received i.v. ondanseiron (8 mg) or equivalent and i.v. lorazepam (2 to 3 mg) pre-treatment, and i.v. metoclopramide (20 mg) post-treatment.

Patients were accrued in two phases: (i) 16 cases began therapy in an initial component between September, 1998 and July, 1999; (ii) 13 cases began therapy in a continuation of the study with the same protocol being used through the entire study.

The primary objectives of the study were to evaluate the pain response rate and to determine the feasibility and safety of administering this combination; secondary endpoints were to assess the duration of the pain responses, quality of life by the EORTC questionnaire, survival benefit, and to determine the PSA response rate. The pain response required a decrease in the Present Pain Intensity (PPI) score on the McGill-Melzack questionnaire in the setting of stable or decreasing analgesic usage (Bureau et al, 1992). PPI scores and analgesic usage were recorded by each patient prior to commencement of therapy and then daily Quality of life assessments were carried out using the EORTC QLQ-30 core questionnaire, version -2, and the EORTC Prostate Cancer Specific Module (PCSM) (Aaronson et al, 1993, King, 1996). Patients completed these indices every three weeks. Because of the difficulties of accurately quantifying response in bone metastases (in the context that tumor-related pain was a requirement for entry), no systematic attempt was made to quantify objective response.

Patients were eligible for this protocol if they met the following criteria: biopsy-proven prostate cancer; symptomatic painful metastases requiring narcotic analgesia (with a PPI score of ≧1); proven castrate testosterone levels (<30 ng/ml); ECOG Performance Status≦2; left ventricular ejection fraction (LVF)>50%; white blood cell count of ≧3.0×10⁹/L, absolute granulocyte count of >1.5×10⁹/L, platelets>100×10⁹/L, and hemoglobin≧9 g/dl; serum creatinine<1.5 times the upper limit of normal, total bilirubin<2.0 times the upper limit of normal and AST<4.0 times the upper limit of normal; documented progression; ani-androgen withdrawal with documented lack of response (>4 weeks for flutamide; >6 weeks for bicalutamide); ≦1 prior chemotherapy regimen, prior radiotherapy (>4 weeks, or strontium 89>8 weeks, before study entry); no prior anthracyclines or anthracenediones; written informed consent.

Patients were excluded if they had a history of prior intercurrent malignancy (apart from treated non-melanoma skin cancer), known central nervous system metastases, acite intercurrent medical disorder, contra-indications to corticosteroids, unstable impending pathological fractures, prior exposure to DPPE, and concurrent participation in other investigational trial.

The characteristics of the treated patients are summarized in Table 1. The median age at presentation was 74 years, with the fourth quartile being 77 years or older. Ten per cent of patients were older than 80 years, suggesting that this series was not confounded by an age selection bias in favor of youth. On the other hand, the majority of the patients had ECOG performance status 1, suggesting case selection in favor of pre-treatment robustness. Despite the good performance status of these patients, the median serum alkaline phosphatase was 344 IU/l and the median PSA was 210 ng/ml (with one quarter having values ≧500 ng/ml), suggesting that this series did not select for minimal metastatic disease. All but one patient had bone metastases, and a small proportion of patients had other sites of soft tissue involvement, including lymph nodes, lung and liver. All patients had pain at entry, which distinguishes this series from most recent reports (See Table 2). The median PPI score at entry was 2 (range 0 to 5). Only one patient self-recorded a PPI score of 0 at entry, although it was documented in the chart by the treating physician that he initially had significant pain, requiring narcotic analgesics, immediately prior to entry in the trial.

### Example II

This Example describes the results of the Phase II clinical trial described in Example I.

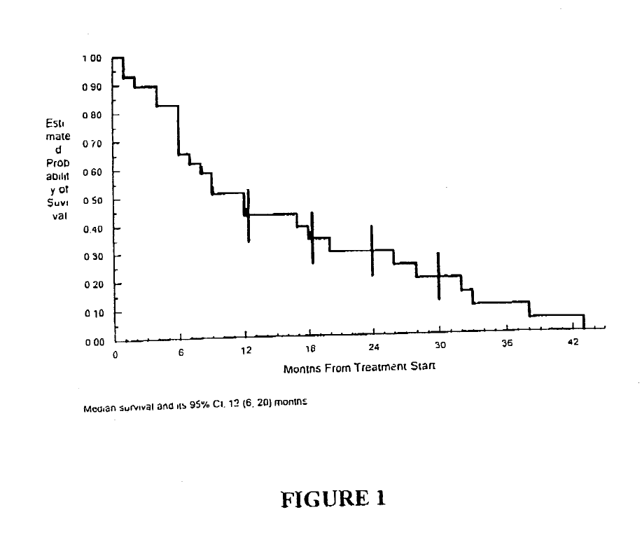

The results of the Phase II clinical trial described in Example I are summarized in Tables 3 and 4 and FIG. 1. As can be seen, 79% of patients sustained a pain response, 66% of patients reduced analgesia, 56% bad a PSA reduction of ≧50% and 48% had a ≧75% PSA reduction The actuarial median survival was 12 months, although it was noted that 2-year survival was 24% and 3-year survival was 10%.

The pattern of pain relief was dramatic. Of the 15 patients with PPI of 2-5, ten reduced by more than 1.5; of the 13 patients with PPI of 1-1.9, five reduced by a PPI score of ≧1. One patient, while complaining of significant pain at baseline, scored himself with a PPI of 0, but was noted to have no pain after two cycles of treatment. In parallel to these results, 11 patients discontinued narcotic analgesics and 8 reduced their use (a total of 66% of cases reduced the use of narcotic analgesia).

The pattern of toxicity is summarized in Table 4. The only unusual side effect was the presence of visual hallucinations, which were attributable to DPPE and seen in 22% of cases, usually in the first cycle only. Apart from hallucinations, Grade III toxicities consisted of asthenia, nausea, vomiting and anorexia. Grade IV leucopenia and thrombocytopenia were noted in one case each. Cardiac toxicity did not appear to be enhanced, although one patient who had received 8 cycles of DPPE/mitoxantrone developed biventricular cardiac failure after receiving indirect cardiac irradiation during radiotherapy for acute spinal cord compression. There were no other cases of cardiac failure, and it should be noted that 60% of patients received >6 cycles, with a range from 2 to 12 cycles of treatment. There were no toxic deaths.

The pattern of five patients experienced serious adverse events defined as possibly related to study drugs. These consisted of hospitalization for fever in one case, severe fatigue in two cases and a possible embolic event in another. These patients recovered or became stable following appropriate treatment. A further patient experienced shortness of breath, weakness and anorexia and treatment was withdrawn at the subject's request. This patient subsequently died.

We have compared outcomes from the Phase II trial described herein with three major previous series (Raghavan et al, 1989, Tannock et al, 1996; Kantoff et al, 1999) to set the general data into a context. While recognizing limitations of non-randomized comparison, in conducting this Phase II trial of 29 patients treated with the combination of mitoxantrone, DPPE and prednisone, we attempted to include only patients that are comparable to those treated in two previous studies (Raghavan et al, 1989; Tannock et al, 1996). While the series of Kantoff et al (1999) is important and instructive, it appears that the patient population was quite different, with a lower tumor burden: 40% of cases did not require analgesia at presentation, only 90% had bone metastases, median PSA was 150 ng/ml and median alkaline phosphatase was 167 IU/l.

We observed a substantial reduction of pain (61%), analgesic consumption (66%), PSA (56% with PSA reduction of ≧50% and 48% with PSA reduction of ≧75%) and serum alkaline phosphatase (36%≧50%). Of particular importance, one quarter of the patients were aged 77 years or older, a population not suited to the more aggressive taxane-estramustine based regimens that have become relatively standard therapy in the younger cohort of patients with prostate cancer.

While the actuarial median survival was not apparently improved (12 months), 28% of cases lived 18 months or longer. 24% lived 2 years or longer, and 10% lived beyond 3 years. These figures are substantially better than previously reported for similar patient populations treated with mitoxantrone plus prednisone (Raghavan et al, 1989; Tannock et al, 1996). The survival data in our study are also of interest in light of the recent phase III randomized National Cancer Institute of Canada study described in the aforementioned PCT/CA02/01725, where patients with advanced breast cancer treated with DPPE/doxorubicin had a significant increase in median survival compared with patients treated with doxorubicin alone. This unusual phenomenon of an apparent increase in the proportion of long-term survivors in the absence of early endpoint differences is currently under investigation in another planned clinical study combining DPPE with an anthracycline. The patients exhibiting longevity all had significant pain symptoms and tended to have high PSA and alkaline phosphates levels, indicative of a high disease burden and poor prognosis for survival beyond one year.

Despite these observations, the pattern of toxicity, listed in Table 4, did not appear significantly different from that previously reported for patients treated with mitoxantrone-prednisone (Raghavan et al, 1989; Tannock et al, 1996; Kantoff et al, 1999), apart from the presence of hallucinations in 10% of cases. The hallucinations have been identified in other cohorts of DPPE-treated cases (Brandes et al, 1995, 1996; Khoo et al. 1999) and are usually described by patients as humorous or pleasant, and are mainly seen with the first cycle of treatment. There have been no patients who have declined to continue treatment because of this phenomenon

The high proportion of patients experiencing 75% PSA reduction and dramatic amelioration of pain suggests that this novel regimen may be more effective than mitoxantrone-prednisone alone, especially when the characteristics of advanced age and extensive bony metastases are considered. Recently, Beer et al (2003) have reported comparable activity of the combination of docetaxel and calcitriol against metastatic, hormone-refractory prostate cancer, although it appears that they have treated patients with a lower tumor burden and less extensive symptoms.

The extent of PSA reduction and decreased symptoms with DPPE/mitoxantrone/prednisone compare favorably to mitoxantrone/prednisone-treated cases reported in the literature, wherein PSA ≧75% reduction occurs in less than 20% of patients and pain reduction in up to 40%. Although median survival is not different from previous experience, improved 2- and 3-year survivals were herein.

### SUMMARY OF DISCLOSURE

In summary of this disclosure, the present invention provides a novel method of treating hormone-refractory prostate cancer by the sequential administration of DPPE and analog compounds and mitoxantrone, to result in increased survival in a proportion of patients. Modifications are possible within the scope of the invention

TABLE 1
PATIENT CHARACTERISTICS:

| | |
|---|---|
| NUMBER OF CASES | 29 |
| AGE | |
| Median | 74 years |
| Range | 52-83 years |
| Interquartile range | 61-77 years |
| ≧80 years | 10% |
| BASELINE ECOG PERFORMANCE STATUS | |
| Median | 1 |
| Mode | 1 |
| Range | 0-2 |
| BASELINE PSA (ng/ml) | |
| Median | 210 |
| Range | 11-3871 |
| interquartile range | 77-593 |
| ≧100 ng/ml | 69% |
| ≧500 ng/ml | 24% |
| BASELINE ALKALINE PHOSPHATASE (U) | |
| Median | 344 |
| Range | 22-3018 |
| >500 U | 34% |
| SITES Of DISEASE | |
| Bone metastases | 97% |
| Liver metastases | 10%? |
| Pulmonary metastases | 10%? |
| Lymph nodes | 10%? |

TABLE 2
COMPARABLE SERIES

| | Raghavan et al | | Tannock et al. | Kantoff et al. | | Beer et al. | |
|---|---|---|---|---|---|---|---|
| INDEX | 1989 | | 1996 | 1999 | | 2003 | Present Series |
| Age | | | | | | | |
| Median | 67 | | 69 | 72 | | 76 | 74 |
| Interquarrile | 50-77* | | 63-75 | 67-75 | | 46-83* | 61-77 |
| range | | | | | | | |
| ECOG P.S. | | | | | | | |
| 0 | | | 6% | | | 35% | 3% |
| | {close oversize brace} | 55% | 57% | {close oversize brace} | 85% | 41% | 83% |
| 1 | | | | | | | |

| ≧2 | 45% | 37% | | 15% | 24% | 14% |
|---|---|---|---|---|---|---|
| Metastases: | | | | | | |
| Bone | 89% | 98% | | 91% | 91% | 97% |
| Lung | 5% | | | 21% | 0 | 10% |
| | | cr,2 | 4% | | | |
| Liver | 8% | | | 9% | 0 | 10% |

| | | | | | |
|---|---|---|---|---|---|
| Nodes | 8% | 22% | 9% | 40% | 10% |
| PSA (ng/ml) | | | | | |
| Median | Not stated | 200 | 150 | 99 | 210 |
| Interquarrile | | 66-678 | 52-362 | 6-921* | 77-593 |
| range | | | | | |
| Alkaline P'ase | | | | | |
| Median | Not stated | 20 | 167 | 127 | 344 |
| Range | | 10-53 | 105-317 | 41-1304* | |
| % with Pain | Not defined | 99% | Not defined | Not defined | 100% |
| % with Narcotic | Not defined | | Not defined | Not defined | |
| analgesics | | | | | |

*total (not interquarrile) range

TABLE 3
RESPONSE AND SURVIVAL:

| PAIN IMPROVED | | |
|---|---|---|
| PPI reduced (total) | | 79% |
| PPI reduced by ≧1 level | (n = 28) | 61% |
| PPI reduced by ≧2 levels | (n = 15) | 47% |

| REDUCTION IN NARCOTIC | | |
|---|---|---|
| ANALGESIA | | |
| Complete | 38% | |
| Partial | 28% | |
| PSA REDUCTION | | |
| ≧50% | 56% | |
| ≧75% | 48% | |
| ALKALINE PHOSPHATASE | | |
| REDUCTION | | |
| ≧50% from baseline of ≧400 U | 36% | |
| ≧50% from baseline of ≧300 U | 36% | |
| ≧50% from baseline of ≧200 U | 32% | |
| SURVIVAL | | |
| Median of all patients | 9.5 | mos |
| Range | 1-43 | mos |

| | |
|---|---|
| ≧18 months | 28% |
| ≧24 months | 24% |
| ≧36 months | 10% |
| Deceased | 90% |
| Alive (mos): 9+, 12+, 18+ | 10% |
| Cause of death | |
| Cancer | 25/29 |
| Myocardial infarction* | 1/29 |

*Patient had active cancer at the time

TABLE 4
TOXICITY
Toxicities Related To DPPE/Mitoxantrone/Prednisone

| | No. of Patients with Toxicity | | | | Patients | |
|---|---|---|---|---|---|---|
| | Grade (n = 28) | | | | with Toxicity | |
| Toxicity | 1 | 2 | 3 | 4 | Total No. | % |
| Gastrointestinal | | | | | | |
| Nausea | 10 | 4 | 2 | | 16 | 57 |
| Vomiting | 7 | 5 | 2 | | 14 | 50 |
| Consitpation | 6 | 5 | 1 | | 12 | 43 |
| Anorexia | 9 | 8 | 2 | | 19 | 68 |
| Metabolic | | | | | | |
| Dehydration | | | | 1 | 1 | 4 |
| Hematologic | | | | | | |
| Leucopenia | | | 1 | 1 | 2 | 7 |
| Thrombocytopenia | | | | 1 | 1 | 4 |
| Musculoskeletal. | | | | | | |
| Bone Pain | 2 | | 1 | | 3 | 10 |
| Myesthenia | 5 | 6 | 2 | | 13 | 46 |
| Neurologic | | | | | | |
| Dizziness | 3 | | | | 3 | 11 |
| Ataxia | 1 | 2 | | | 3 | 11 |
| Hallucinations** | | | 6 | | 6 | 22 |
| Insomnia | 1 | 3 | 2 | | 6 | 22 |
| Depression | | 4 | 1 | | 5 | 18 |
| Body as a whole | | | | | | |
| Asthenia | 3 | 10 | 8 | | 21 | 75 |

**All episodes of hallucination are graded as III

References

Aaronson N K, Ahmedsai S, Bergman B et al: The European Organization for Research and Treatment of Cancer QLQ-30. A quality-of-life instrument for use in international clinical trials in oncology. J. Natl. Cancer Inst., 1993, 85: 365-376.

Beer T M, Eilers K M, Garzonto M, Egorin M J, Lowe B A, and Henner W D: Weekly high-dose calcitriol and docetaxel in metastatic androgen-independent prosrate cancer. J. Clin Oncol., 2003, 21: 123-128.

Brandes L J, Bogdanovic R P, Cawker M D et al: Histamine and growth; interaction of antiestrogen binding site ligands with a novel histamine site that may be associated with calcium channels. Cancer Res , 1987, 47: 4025-4031.

Brandes L J, La Bella F S, Warrington R C: Increased therapeutic index of antineoplastic drugs in combination with intracellular histamine antagonists. J. Natl. Cancer Inst. 1991, 83: 1329-1336.

Brandes L J, Simons K J, Bracken S P, Warrington R C: Results of a clinical trial in humans with refractory cancer of the intracellular histamine antagonist, N,N-diethyl-2-4[4-(phenylmethyl) phenoxy]ethanamine-HCl, in combination with various single antineoplastic agents. J. Clin. Oncol., 1994, 12:??? 1281-1290.

Brandes L J, Bracken S P, Ramsey E W. N,N-diethyl-2-[4-(phenylmethyl)phenoxy [ethanamine in combination with cyclophosphamide: An active low-toxicity regimen for metastatic hormonally unresponsive prostate cancer. J. Clin. Oncol, 1995, 12. 1398-1403.

Brandes L J, Bracken S P. The intracellular histamine antagonist, N,N-diethyl-2-[4-(phenylmethyl)phenoxyl]ethamine.HCL, may potentiate doxorubicin in the treatment of metastatic breast cancer: Results of a pilot study. Breast Cancer Res Treat. 1998, 49; 61-68.

Brandes L J, Queen G M, LaBella F S N,N-diethyl-[4-(phenylmethyt)phenoxy]ethanamine (D)PPE) a chemopoientiating and cytoprotective agent in clinical trials: interaction with histamine at cytochrome P450 3A4 and other isozymes that metabolize antineoplastic drugs. Cancer Chemother Pharmacol. 2000; 45:298-304.

Bureau F et al: comparative study of the validity of four French McGill Pain Questionnaire (MPQ) versions. Pain, 1992, 50; 59-95.

Kantoff P W, Halabi S, Conadway M et al: Hydrocortisone with or without mitoxantrone in men with hormone-refractory prostate cancer; Results of the Cancer and Leukemia Group B 9182 study. J. Clin. Oncol., 1999, 17: 2506-2513.

Khoo K, Brandes L, Reyno L et al. Phase II trial of N,N-diethyl-2-[4-(phenylmethyl)phenoxy]ethanamine. HCl and doxorubicin chemotherapy in metastatic breast cancer. A National Cancer Institute of Canada clinical trials group study. J Clin Oncol 1999; 17: 3431-3437.

King M T: The interpretation of scores from the EORTC quality-of-life questionnaire QLQ-C30. Quality of Life Research, 1996, 5: 555-576.

Menendez A T, Raventos-Suarez C, Fairchild C et al. Mechanism of action of DPPE, a chemosensitizing agent Proc. Amer. Assoc, Cancer Res. 1998, 39 3462 (Abstr.)

Osborne C K, Drelichman A, Von Hoff D D, Crawford E D: Mitoxatrone Modest activity in a phase II trial in advanced prostate cancer. Cancer Treat. Rep, 1983, 57: 1133-1135.

Raghavan D, Pearson B, Coorey G, et al. Management of hormone-resistant prostate cancer: Experience at Royal Prince Alfred Hospital. In: Johnson D E, Samuels M, Logothetis C J (eds), Systemic Therapy for Genitourinary Cancers, Year Book Publishers, Chicago, pp 245-250, 1989.

Tannock I F, Osoba D, Stockler M R et al: Chemotherapy with mitoxantrone plus prednisone or prednisone alone for symptomatic hormone-resistant prostate cancer: A Canadian randomized trial with palliative end points. J Clin. Oncol, 1996, 14: 1756-1764.

What I claim is:

1. A method of achieving enhanced survival in a proportion of human patients with hormone-refractory prostate cancer, which comprises:
(a) first administering to said patients at least one diphenyl compound of the formula: 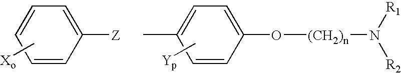
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each an alkyl group containing 1 to 3 carbon atoms or are joined together to form a heterocyclic ring with the nitrogen atom and n is 1, 2 or 3, or pharmaceutically-acceptable salts thereof, and
(b) following sufficient time to permit inhibition of binding of intracellular histamine, subsequently administering to the patient a chemotherapeutic agent active in hormone-refractory prostate cancer.

2. The method of claim 1 wherein the group 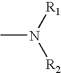
is a diethylamino groups a dimethylamino group, a morpholino group or a piperazino group.

3. The method of claim 1 wherein the group 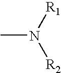
is a diethylamino group, Z is —CH₂, n is 2 and o and p are each 0.

4. The method of claim 3 wherein diphenyl compound is in the form of a hydrochloride salt or free base.

5. The method of claim 1 wherein said chemotherapeutic agent active in hormone-refractory prostate cancer is mitoxantrone.

6. The method of claim 1 wherein said diphenyl compound is administered to the patient about 30 to about 90 minutes prior to said administration of said chemotherapeutic agent.

7. The method of claim 6 wherein said time is about 60 minutes.

8. The method of claim 5 wherein said diphenyl compound is administered by intravenous infusion of a solution thereof over a period of time of up to about 90 minutes prior to administration of said chemotherapeutic agent and is maintained during administration of said chemotherapeutic agent.

9. The method of claim 8 wherein said diphenyl compound is administered for about 60 minutes prior to administration of said chemotherapeutic agent and is maintained during about 20 minutes intravenous infusion of said chemotherapeutic agent.

10. The method of claim 6 wherein said diphenyl compound is administered in an amount of about 8 to about 240 mg/M² of said patient.

11. The method of claim 10 wherein said amount is about 3 to about 10 mg/kg of patient.

12. The method of claim 8 wherein said diphenyl compound is administered in an amount of about 3 to about 10 mg/kg of patient.

13 The method of claim 9 wherein said diphenyl compound is administered in an amount of about 6 mg/kg in the form of the hydrochloride salt or 5.3 mg/kg in the form of the free base.

14. The method of claim 10 wherein said chemotherapeutic agent is administered in an amount of about 50 to about 75 mg/M² of patient.

15. The method of claim 13 wherein said chemotherapeutic agent is administered in an amount of about 60 mg/M² of patient.
